(19) United States
(12) Patent Application Publication
(10) Pub. No.: US 2025/0267783 A1
(43) Pub. Date: Aug. 21, 2025

(54) Y-TYPE INCLINED LITHIUM TARGET FOR GENERATING HIGH POWER NEUTRONS

(71) Applicant: KOREA INSTITUTE OF RADIOLOGICAL & MEDICAL SCIENCES, Seoul (KR)

(72) Inventors: Bong Hwan HONG, Seoul (KR); Min Ho KIM, Seoul (KR); Seung Woo PARK, Seoul (KR); Cha Won PARK, Hwaseong-si (KR)

(21) Appl. No.: 18/702,966

(22) PCT Filed: Nov. 16, 2022
(86) PCT No.: PCT/KR2022/018096

§ 371 Date: Apr. 19, 2024

(30) Foreign Application Priority Data
Nov. 30, 2021 (KR) 10-2021-0169025

(51) Int. Cl. H05H 6/00; A61N 5/10; G21G 4/02

(52) U.S. Cl. CPC H05H 6/00; A61N 5/10; G21G 4/02; A61N 2005/109; H05H 2242/10; H05H 2277/116

(57) ABSTRACT

The present invention relates to a y-type inclined lithium target for generating high power neutrons, comprising a pair of target modules comprising a substrate extending to a predetermined width and a solid target provided on one surface of the substrate, wherein the pair of target modules are installed at a first angle with respect to each other and are disposed at a second angle with respect to a direction in which a particle beam is irradiated. The y-type inclined lithium target for generating high power neutrons according to the present invention has the advantage of being capable of maximizing a heat transfer area. In addition, since the target is configured as a pair of target modules, each of the target modules can be miniaturized. Accordingly, generation and accuracy of a lithium layer can be improved, and the advantage of easy storage is provided.

### CROSS-REFERENCE TO RELATED APPLICATIONS

This application is the National Stage filing under 35 U.S.C. 371 of International Application No. PCT/KR2022/018096, filed on Nov. 16, 2022, which claims the benefit of Korean Patent Application No. 10-2021-0169025 filed on Nov. 30, 2021, the contents of which are all hereby incorporated by reference herein in their entirety.

### TECHNICAL FIELD

The present disclosure relates to a y-type inclined lithium target for generating high power neutrons with increased heat transfer area and increased cooling efficiency.

### BACKGROUND ART

Neutron capture therapy involves administering materials containing boron in advance to accumulate boron in cancer cells, then irradiating them with neutrons to induce nuclear fission within the cancer cells, and finally destroying the cancer cells as particles are emitted due to the fission. Boron neutron capture therapy is known to be effective for brain tumors, head and neck cancer, skin cancer, and is gaining attention as a next-generation cancer treatment method due to its ability to minimize side effects caused by radiation exposure to normal cells compared to conventional radiation therapy methods.

The neutron generating device is composed of a target, including a target that emits neutrons rapidly colliding with a positive ion beam, placed along the beam path of a particle accelerator such as a cyclotron.

When the target is irradiated with a positive ion beam, it heats up due to nuclear reactions, necessitating a cooling process. The US Patent US20170062086 discloses conventional technology related to this.

But it has been noted that conventional technology has difficulties in target replacement, excessive heating when irradiating high-power particle beams, and low cooling efficiency.

### DISCLOSURE

### Technical Problem

The purpose of the present invention is to provide a y-type inclined lithium target for generating high power neutrons to solve the problems of the neutron generation target used in conventional boron neutron capture therapy.

### Technical Solution

To solve the aforementioned problem, the present disclosure provides y-type inclined lithium target for generating high power neutrons can be provided, which comprises comprising the following, a substrate formed to extend to a predetermined area and a pair of target modules including a solid target provided on one side of the substrate, wherein the pair of target modules are installed at a first angle to each other, and are arranged at a second angle to the direction in which the particle beam is irradiated.

Meanwhile, a pair of target modules may be configured such that a portion of each target module is disposed on the irradiation path of the particle beam.

Additionally, the pair of target modules may rotate around an axis perpendicular to the irradiation path of the particle beam to form a second angle.

Meanwhile, each target module may be configured as a flat plate extending in one direction.

Additionally, a pair of target modules may be arranged side by side along the direction in which each plate extends, and may be arranged at a first angle to each other about an axis parallel to the direction in which each plate extends.

Meanwhile, the first angle may be 180 degrees or less, and the second angle may be 90 degrees or less.

In addition, the pair of target modules includes a first target module and a second target module, and adjacent ends of the first target module and the second target module may be arranged to be offset.

Furthermore, the first target module and the second target module may be arranged so that a portion of the particle beam irradiated once is irradiated to the first target module, and the remainder of the particle beam irradiated once is irradiated to the second target module.

Meanwhile, the heat transfer area of the first target module and the heat transfer area of the second target module may be formed asymmetrically.

Meanwhile, the first target module and the second target module may each include a lithium layer provided on the surface to which the particle beam is irradiated.

Meanwhile, the lithium layer may be formed by plating or depositing on one side of the substrate.

Additionally, the first target module and the second target module may be configured so that the surface opposite to the surface on which the particle beam is irradiated can be cooled.

### Advantageous Effects

The y-type inclined lithium target for generating high power neutrons according to the present invention can improve cooling efficiency by increasing the heat transfer area, and thus can further increase the beam power of protons, thereby further increasing the yield of neutrons.

### DESCRIPTION OF DRAWINGS

FIG. 1 is a diagram illustrating the concept of boron neutron capture therapy.

FIG. 2 is a diagram showing a state in which a y-type inclined lithium target for generating high power neutrons according to the present invention is installed.

FIG. 3 is a cross-sectional view of the target module of the present invention.

FIG. 4 is a perspective view of a y-type inclined lithium target for generating high power neutrons.

FIG. 5 is a diagram showing a y-type inclined lithium target for generating high-power neutrons along the direction in which a particle beam is irradiated.

FIG. 6 is a cross-sectional view of a y-type inclined lithium target for generating high power neutrons.

FIGS. 7A, 7B, and 7C are diagrams showing the heat transfer area in various planar neutron generation targets.

### MODE FOR DISCLOSURE

Hereinafter, y-type inclined lithium target for generating high power neutrons according to embodiments of the present disclosure will be described in detail with reference to the attached drawings. In the description of the following embodiments, the name of each component may be referred to as different names in the art. However, if there is functional similarity and identity between components, they can be regarded as equivalent components even if modified embodiments are adopted. Additionally, a symbol is attached to each component for convenience of description. However, content shown in the drawings in which such symbols are written does not limit each component to the scope within the drawings. Likewise, even if an embodiment in which a configuration in a drawing is partially modified is adopted, it can be regarded as an equivalent configuration if there is functional similarity and identity. Further, if a component is recognized as a component that should be included in light of the general level of technicians in the relevant technical field, the description thereof will be omitted.

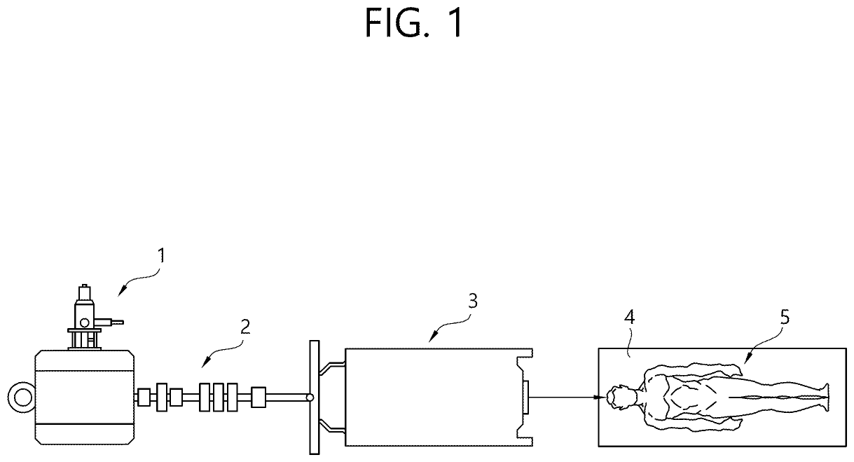

FIG. 1 is a diagram illustrating the concept of boron neutron capture therapy.

As shown, the neutron generator that generates neutrons in the conventional boron neutron capture treatment is a particle accelerator 1 such as a cyclotron, linear accelerator, and electrostatic accelerator, and a proton beam emitted at high speed from the particle accelerator 1 is accelerated. It consists of an electrostatic accelerator 2 and a target that is installed on the beam path of the proton beam and collides with the beam to emit neutrons therein.

Neutrons generated from the target can be divided into fast neutrons with an energy of 10 keV or more, epithermal neutrons from 0.5 eV to 10 keV, and thermal neutrons with an energy of 0.5 eV or less. The beam shaping device 3 is divided so that it can be converted into extrathermal neutrons suitable for treatment.

The neutron beam that has passed through the beam shaping device 3 is configured to pass through a desired area by a collimator, and is finally irradiated to the affected area of the patient 5 to cause a nuclear reaction.

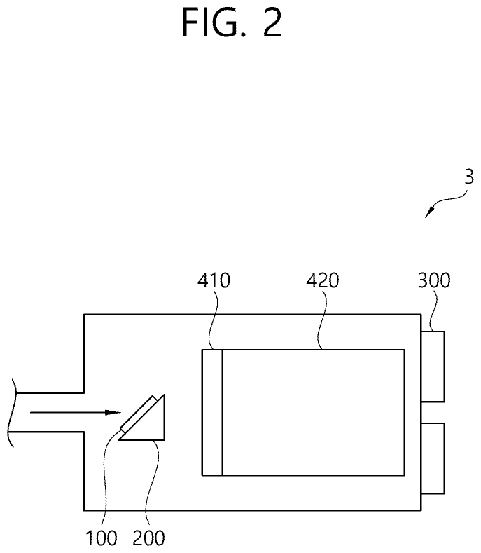

FIG. 2 is a diagram showing a state in which a y-type inclined lithium target for generating high power neutrons according to the present invention is installed.

Referring to FIG. 2, the y-type inclined lithium target 100 for generating high-power neutrons according to the present invention is provided in a vacuum chamber and can be placed on the irradiation path of the proton beam. Additionally, the target may be in contact with the cooling unit 200 to discharge heat generated when the particle beam is irradiated.

The proton beam irradiated to the target 100 undergoes a nuclear reaction with lithium, and at this time, the emitted neutrons pass through the fast neutron filter 410, the moderator 420, and the collimator 300 and are finally irradiated to the patient's affected area.

One of the wide surfaces of the y-type inclined lithium target 100 for generating high power neutrons according to the present invention (hereinafter referred to as ‘front surface’) is exposed to the irradiation path of the proton beam, and the opposite wide surface (hereinafter referred to as ‘back surface’) is cooled. It is configured to allow cooling in association with the unit 200.

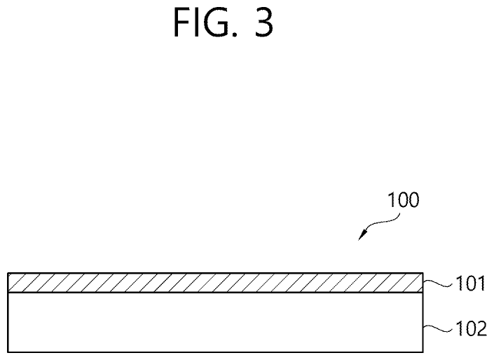

Hereinafter, the configuration of the y-type inclined lithium target for generating high power neutrons according to the present invention will be described in detail with reference to FIGS. 3 to 7C.

FIG. 3 is a cross-sectional view of the target module of the present invention. In this drawing, the thickness of the lithium layer 101 is somewhat exaggerated to facilitate understanding. However, the lithium layer 101 may be formed to have a thickness of several tens of μm.

The y-type lithium target 100 for generating high power neutrons according to the present invention is composed of a pair of planar target modules so that a proton beam can be irradiated. The target module may be configured as a flat plate extending in one direction. That is, the target module may be configured in a rectangular shape on a plane. The target module 100 may include a flat substrate 102 and a lithium layer 101. The substrate 102 may be made of a metal material so that heat generated in the lithium layer 101 and the substrate 102 can be easily transferred to the outside when irradiated with a proton beam. As an example, the substrate 102 may include copper.

The lithium layer 101 may be provided on one wide surface of the substrate. A lithium layer may be provided on a substrate using processes such as plating and deposition.

As described above, the target 100 used in the present invention may use a pair of target modules of the identical structure.

In order to secure the neutron yield required for neutron capture treatment, the target must be able to handle a high output beam power of 30 kW or more. In particular, when the target includes a lithium layer, it is desirable to expand the heat transfer area because the melting point of lithium is 180.5° C. Therefore, in order to expand the heat transfer area and improve cooling performance, a flat solid target is installed at an angle to the path of the particle beam.

Meanwhile, it is more advantageous in terms of process to create a target module with divided areas than when it is composed of a single flat module with a large area because it satisfies ease of creation of the lithium layer and uniformity of thickness. Additionally, a target module with a small area can reduce the required area on a plane even when stored after use.

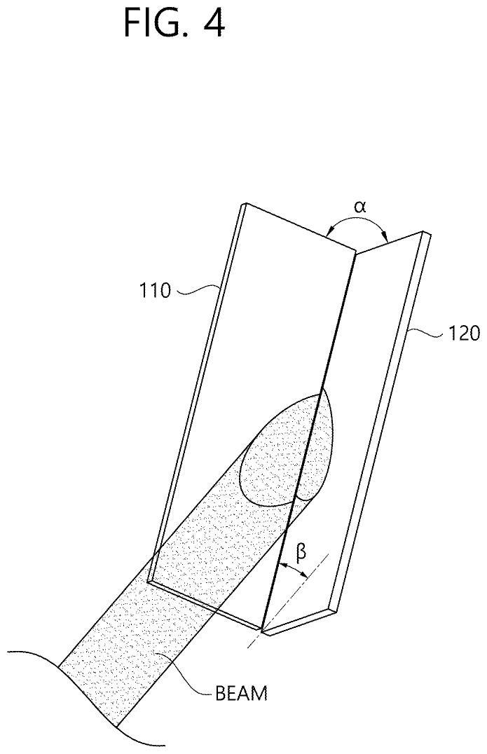

Hereinafter, with reference to FIGS. 4 to 6, a detailed description will be given of the state in which the y-type inclined lithium target for generating high power neutrons according to the present invention is placed in the chamber.

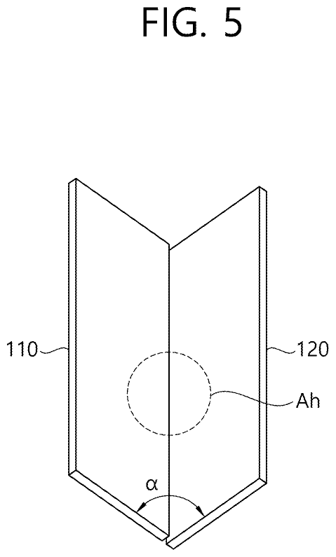

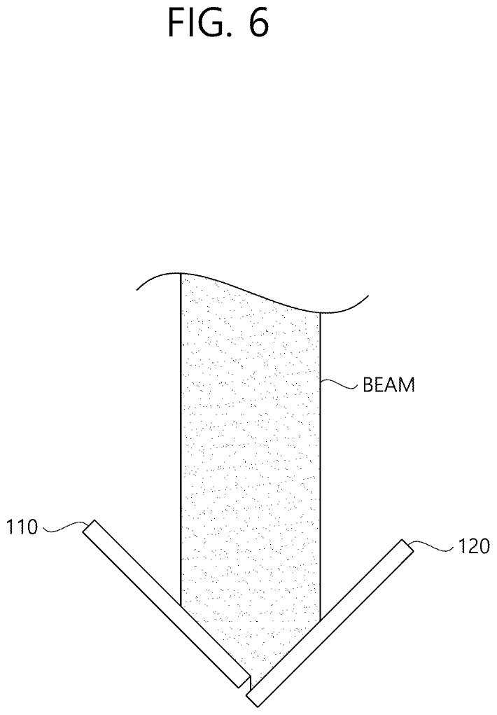

FIG. 4 is a perspective view of a y-type inclined lithium target for generating high power neutrons, FIG. 5 is a diagram showing a y-type inclined lithium target for generating high power neutrons along the direction in which the particle beam is irradiated and FIG. 6 is a cross-sectional view of a y-type inclined lithium target for generating high power neutrons.

As shown in FIGS. 4 to 6, the y-type inclined lithium target for generating high power neutrons according to the present invention can be used together with a pair of target modules. Hereinafter, for convenience, the pair of target modules will be described by dividing them into the first target module 110 and the second target module 120.

The ends of the first target module 110 and the second target module 120 are arranged to be offset from each other. Referring again to FIG. 6, the first target module 110 and the second target module 120 may have a y-type cross-section when disposed.

Additionally, the first target module 110 and the second target module 120 may be arranged to be spaced apart from each other at a first angle α. The first angle α may be 180 degrees or less. That is, the first target module 110 and the second target module 120 may be arranged at an angle of 180 degrees or less so that the heat transfer area Ah can be expanded compared to when they are arranged side by side on a plane. In one embodiment, the first angle α may be 90 degrees.

That is, the side surface of the first target module 110 is arranged to be in close contact with the lithium layer of the second target module 120. At this time, the distance between the first target module 110 and the second target module 120 is determined so that the particle beam can be irradiated to both target modules. Therefore, the particle beam irradiated once is distributed to the first target module 110 and the second target module 120 to cause a nuclear reaction.

Meanwhile, the first target module 110 may be arranged so that the area where the lithium layer of the first target module 110 and the lithium layer of the second target module 120 overlap in the particle beam irradiation path can be minimized. As described above, in the path of the particle beam, the lithium layer of the first target module 110 overlaps the lithium layer of the second target module 120 in a certain area. In order to maintain a constant quality of neutrons generated at this time, it is desirable to minimize the number of overlapping lithium layers. Accordingly, the first target module 110 and the second target module 120 are arranged in a y-shape, but may be arranged in a form close to a v-shape as shown in FIGS. 4 to 6.

Referring again to FIG. 4, the first target module 110 and the second target module 120 are arranged at an angle to each other with a second angle β centered on an axis orthogonal to the irradiation direction of the particle beam. That is, the first target module 110 and the second target module 120 may be arranged in an inclined state while facing the acceleration view. As an example, when the particle beam is irradiated in the horizontal direction, the first target module 110 and the second target module 120 may be arranged at a second angle β in the horizontal direction. The second angle β may be determined to be within 90 degrees from the irradiation direction of the particle beam. In one embodiment, the second angle β may be determined to be 45 degrees.

Meanwhile, although not shown, the first target module 110 and the second target module 120 are configured to be detachable in a y-shape in the chamber, and the first target module 110 and the second target module 120 A cooling unit capable of cooling may be provided. When the first target module 110 and the second target module 120 are arranged in a y-shape, at least a portion of the rear surface of each is configured to contact the cooling part to transfer heat.

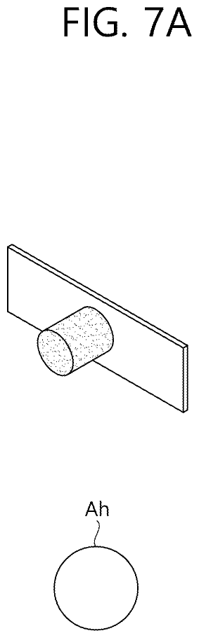

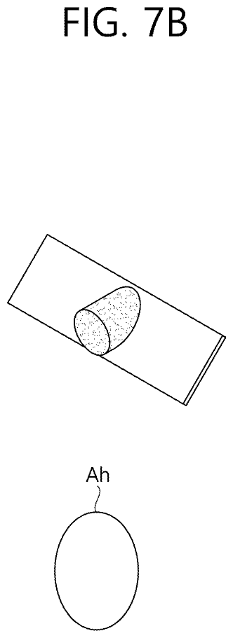

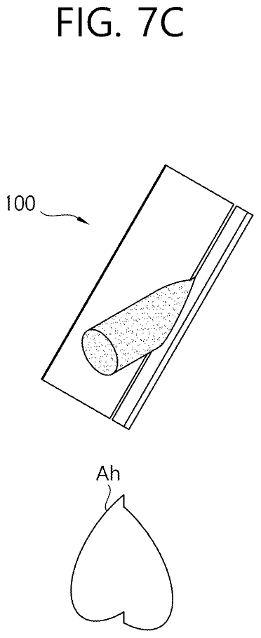

Hereinafter, the heat transfer area formed on each target when irradiated with a particle beam under the same conditions will be described with reference to FIGS. 7A, 7B, and 7C.

FIGS. 7A, 7B, and 7C are diagrams showing the heat transfer area in various planar neutron generation targets.

Referring to FIG. 7A, when a plane is formed in a direction perpendicular to the irradiation path of the particle beam, the heat transfer area becomes circular. At this time, the heat transfer area Ah is the smallest and the temperature becomes relatively high.

Referring to FIG. 7B, when a flat target is tilted in one direction, the heat transfer area appears in an oval shape. Although the heat transfer area Ah is increased compared to the state shown in FIG. 7A, there is a limit to maintaining the temperature below an appropriate temperature when a high-power particle beam is irradiated.

Referring to the top of FIG. 7C, a state in which a particle beam is irradiated to a pair of target modules 100, which is an embodiment according to the present invention, is shown. Additionally, at the bottom of FIG. 7C, the heat transfer area is shown when a pair of target modules 100 are arranged in parallel on a plane. In the wye-type inclined lithium target for generating high power neutrons according to the present invention, a pair of target modules 100 are arranged to be offset from each other, so that the heat transfer area Ah formed in each target module can be formed asymmetrically, the heat transfer area Ah can be maximized.

As described above, the y-type inclined lithium target for generating high power neutrons according to the present invention is provided with an angle adjusted in two axes from the irradiation path of the particle beam, which has the effect of maximizing the heat transfer area. Additionally, since the target consists of a pair of target modules, each target module can be miniaturized. Therefore, the creation and accuracy of the lithium layer can be improved, and storage is easy.

1. A y-type inclined lithium target for generating high power neutrons comprising;
a pair of target modules including a substrate extending to a predetermined width and a solid target provided on one surface of the substrate;
wherein the pair of target modules is,
installed at a first angle to each other,
placed at a second angle to the direction in which the particle beam is irradiated.

2. The y-type inclined lithium target for generating high power neutrons according to claim 1, wherein the pair of target modules is,
configured so that a portion of each target module is disposed on the irradiation path of the particle beam.

3. The y-type inclined lithium target for generating high power neutrons according to claim 2, wherein the pair of target modules are angled about an axis perpendicular to the irradiation path of the particle beam to form the second angle.

4. The y-type inclined lithium target for generating high power neutrons according to claim 3, wherein the each of the target modules is configured as a flat plate extending in one direction.

5. The y-type inclined lithium target for generating high power neutrons according to claim 4, wherein the pair of target modules is,
placed side by side along the extending direction,
disposed at the first angle with respect to an axis parallel to the extended direction.

6. The y-type inclined lithium target for generating high power neutrons according to claim 5, wherein the first angle is 180 degrees or less, and
the second angle is 90 degrees or less.

7. The y-type inclined lithium target for generating high power neutrons according to claim 2, wherein the pair of target modules is including a first target module and a second target module,
wherein the first target module and the second target module are arranged with adjacent ends offset from each other.

8. The y-type inclined lithium target for generating high power neutrons according to claim 7, wherein the first target module and the second target module are,
where a portion of the particle beam irradiated once is directed onto the first target module, and the remainder of the particle beam irradiated once is arranged to be directed onto the second target module.

9. The y-type inclined lithium target for generating high power neutrons according to claim 8, wherein a heat transfer area of the first target module and a heat transfer area of the second target module are formed asymmetrically.

10. The y-type inclined lithium target for generating high power neutrons according to claim 8, wherein the first target module and the second target module are each including a lithium layer provided on the surface to which the particle beam is irradiated.

11. The y-type inclined lithium target for generating high power neutrons according to claim 10, wherein the lithium layer is formed by plating or depositing on one side of a substrate.

12. The y-type inclined lithium target for generating high power neutrons according to claim 10, wherein the first target module and the second target module are configured to cool the surface opposite to the surface on which the particle beam is irradiated.
